(19) United States
(12) Patent Application Publication
(10) Pub. No.: US 2026/0232438 A1
(43) Pub. Date: Aug. 13, 2026

(54) PROSTHETIC HEART VALVE

(71) Applicant: Edwards Lifesciences Corporation, Irvine, CA (US)

(72) Inventors: Brendan Michael Dalbow, Oceanside, CA (US); Justin Mark Nubla, Irvine, CA (US)

(73) Assignee: Edwards Lifesciences Corporation, Irvine, CA (US)

(21) Appl. No.: 19/644,245

(22) Filed: Apr. 10, 2026

Related U.S. Application Data
(63) Continuation of application No. PCT/US2024/051502, filed on Oct. 16, 2024.
(60) Provisional application No. 63/544,469, filed on Oct. 17, 2023.

(51) Int. Cl. A61F 2/24

(52) U.S. Cl. CPC A61F 2/2418; A61F 2/2415; A61F 2210/0014; A61F 2220/0033; A61F 2230/0006; A61F 2240/001

(57) ABSTRACT

A prosthetic heart valve can include a frame and a leaflet assembly. The frame can include first and second commissure support portions defining a frame opening therebetween. The leaflet assembly can include a first leaflet comprising a first commissure tab, a second leaflet comprising a second commissure tab, a first wedge coupled to the first commissure tab of the first leaflet, and a second wedge coupled to the second commissure tab of the second leaflet. The first and second wedges and the first and second commissure tabs can extend through the frame opening. The first wedge can be coupled to the first commissure support portion and the second wedge can be coupled to the second commissure support portion.

### CROSS-REFERENCE TO RELATED APPLICATION

This application is a continuation of PCT Application No. PCT/US2024/051502, filed October 16, 2024, which claims the benefit of U.S. Provisional Patent Application No. 63/544,469, filed on October 17, 2023, wherein each of the above-referenced applications is incorporated by reference herein in its entirety.

### FIELD

The present disclosure relates to leaflet assemblies for prosthetic heart valves.

### BACKGROUND

The human heart can suffer from various valvular diseases. These valvular diseases can result in significant malfunctioning of the heart and ultimately require repair of the native valve or replacement of the native valve with an artificial valve. There are a number of known repair devices (for example, stents) and artificial valves, as well as a number of known methods of implanting these devices and valves in humans. Percutaneous and minimally-invasive surgical approaches are used in various procedures to deliver prosthetic medical devices to locations inside the body that are not readily accessible by surgery or where access without surgery is desirable. In one specific example, a prosthetic heart valve can be mounted in a crimped state on the distal end of a delivery apparatus and advanced through the patient’s vasculature (for example, through a femoral artery and the aorta) until the prosthetic heart valve reaches the implantation site in the heart. The prosthetic heart valve is then expanded to its functional size, for example, by inflating a balloon on which the prosthetic valve is mounted, actuating a mechanical actuator that applies an expansion force to the prosthetic heart valve, or by deploying the prosthetic heart valve from a sheath of the delivery apparatus so that the prosthetic heart valve can self-expand to its functional size.

A need exists for prosthetic heart valves that are durable and easy to assemble.

### SUMMARY

Described herein are prosthetic heart valves, leaflet assemblies for prosthetic heart valves, and methods for assembling prosthetic heart valves. The disclosed prosthetic heart valves, leaflet assemblies, and methods can, for example, provide for a durable, easy-to-assemble connection between a frame of a prosthetic heart valve and a leaflet assembly of the prosthetic heart valve. As such, the devices and methods disclosed herein can, among other things, overcome one or more of the deficiencies of typical prosthetic heart valves.

A prosthetic heart valve can comprise a frame and a valvular structure coupled to the frame. In addition to these components, a prosthetic heart valve can further comprise one or more of the components disclosed herein.

In some examples, a prosthetic heart valve can comprise an annular frame and a valvular structure.

In some examples, the frame can comprise first and second commissure support portions defining a frame opening therebetween.

In some examples, the valvular structure can comprise a first leaflet and a second leaflet, the first leaflet comprising a first commissure tab and the second leaflet comprising a second commissure tab.

In some examples, a first clip can be coupled to the first commissure tab.

In some examples, a second clip can be coupled to the second commissure tab.

In some examples, the first clip can be configured to clip onto the first commissure support portion of the frame, and the second clip can be configured to clip onto the second commissure support portion of the frame.

In some examples, the first and second clips form snap-fit connections with the first and second commissure support portions, respectively.

In some examples, each of the first and second clips comprises an outer portion, an inner portion, and an intermediate portion disposed therebetween.

In some examples, the outer portion of each clip is sutured to the corresponding commissure tab.

In some examples, the first and second leaflets are made of a first material and the first and second clips are made of a second material.

In one representative example, a prosthetic heart valve can comprise a frame and a leaflet assembly. The frame can comprise first and second commissure support portions defining a frame opening therebetween. The leaflet assembly can comprise a first leaflet comprising a first commissure tab, a second leaflet comprising a second commissure tab, a first wedge coupled to the first commissure tab of the first leaflet, and a second wedge coupled to the second commissure tab of the second leaflet. The first and second wedges and the first and second commissure tabs can extend at least partially through the frame opening, the first wedge can be coupled to the first commissure support portion, and the second wedge can be coupled to the second commissure support portion.

In one representative example, a prosthetic heart valve can comprise an annular frame and a valvular structure comprising a plurality of leaflets. Each leaflet can have opposing commissure tabs. A clip can be secured to each commissure tab. Each clip can be secured to a portion of the frame with a snap-fit connection.

In one representative example, a valvular structure for a prosthetic heart valve can comprise a first leaflet comprising a first commissure tab disposed on a first side of the first leaflet, a second leaflet comprising a second commissure tab disposed on a second side of the second leaflet, a first clip coupled to the first commissure tab of the first leaflet, wherein the first clip can be configured to clip onto a first commissure support portion of a frame of the prosthetic heart valve, and a second clip coupled to the second commissure tab of the second leaflet, wherein the second clip can be configured to clip onto a second commissure support portion of the frame of the prosthetic heart valve.

In one representative example, a method of assembling a prosthetic valve can comprise coupling a first clip to a first commissure tab of a first leaflet, coupling a second clip to a second commissure tab of a second leaflet, and inserting the first commissure tab of the first leaflet and the second commissure tab of the second leaflet through a commissure window of a prosthetic heart valve frame such that the first commissure tab and the second commissure tab are disposed between the first clip and the second clip.

The various innovations of this disclosure can be used in combination or separately. This summary is provided to introduce a selection of concepts in a simplified form that are further described below in the detailed description. This summary is not intended to identify key features or essential features of the claimed subject matter, nor is it intended to be used to limit the scope of the claimed subject matter. The foregoing and other objects, features, and advantages of the disclosure will become more apparent from the following detailed description, claims, and accompanying figures.

### BRIEF DESCRIPTION OF THE DRAWINGS

FIG. 1 is a perspective view of an exemplary prosthetic heart valve.

FIG. 2 is a perspective view of an exemplary frame of a prosthetic heart valve.

FIG. 3A is an enlarged, perspective view of a portion of an exemplary prosthetic heart valve.

FIG. 3B is an enlarged side view of a portion of the prosthetic heart valve of FIG. 3A, shown partially in section.

FIG. 3C is an enlarged, cross-sectional view of a portion of the prosthetic heart valve of FIGS. 3A-3B.

FIG. 4 is a side view an exemplary leaflet for the prosthetic heart valve of FIGS. 3A-3C.

FIG. 5A is a perspective view an exemplary shim for a commissure assembly of the prosthetic heart valve of FIGS. 3A-3C.

FIG. 5B is a front view the shim of FIG. 5A.

FIG. 5C is a side view the shim of FIG. 5A.

FIG. 6 is an enlarged, cross-sectional view of a portion of the exemplary prosthetic heart valve of FIGS. 3A-3C.

FIG. 7 is a side view of an exemplary delivery device for a prosthetic heart valve.

### DETAILED DESCRIPTION

### General Considerations

For purposes of this description, certain aspects, advantages, and novel features of examples of this disclosure are described herein. The disclosed methods, apparatus, and systems should not be construed as being limiting in any way. Instead, the present disclosure is directed toward all novel and nonobvious features and aspects of the various disclosed examples, alone and in various combinations and sub-combinations with one another. The methods, apparatus, and systems are not limited to any specific aspect or feature or combination thereof, nor do the disclosed examples require that any one or more specific advantages be present or problems be solved.

Although the operations of some of the disclosed examples are described in a particular, sequential order for convenient presentation, it should be understood that this manner of description encompasses rearrangement, unless a particular ordering is required by specific language set forth below. For example, operations described sequentially may in some cases be rearranged or performed concurrently. Moreover, for the sake of simplicity, the attached figures may not show the various ways in which the disclosed methods can be used in conjunction with other methods. Additionally, the description sometimes uses terms like “provide” or “achieve” to describe the disclosed methods. These terms are high-level abstractions of the actual operations that are performed. The actual operations that correspond to these terms may vary depending on the particular implementation and are readily discernible by one of ordinary skill in the art.

As used in this application and in the claims, the singular forms “a,” “an,” and “the” include the plural forms unless the context clearly dictates otherwise. Additionally, the term “includes” means “comprises.” Further, the term “coupled” generally means physically, mechanically, chemically, magnetically, and/or electrically coupled or linked and does not exclude the presence of intermediate elements between the coupled or associated items absent specific contrary language.

As used herein, the term “proximal” refers to a position, direction, or portion of a device that is closer to the user and further away from the implantation site. As used herein, the term “distal” refers to a position, direction, or portion of a device that is further away from the user and closer to the implantation site. Thus, for example, proximal motion of a device is motion of the device away from the implantation site and toward the user (for example, out of the patient’s body), while distal motion of the device is motion of the device away from the user and toward the implantation site (for example, into the patient’s body). The terms “longitudinal” and “axial” refer to an axis extending in the proximal and distal directions, unless otherwise expressly defined.

As used herein, “e.g.” means “for example,” and “i.e.” means “that is.”

### Examples of the Disclosed Technology

Prosthetic valves disclosed herein can be radially compressible and expandable between a radially compressed state and a radially expanded state. Thus, the prosthetic valves can be crimped on or retained by an implant delivery apparatus in the radially compressed state during delivery, and then expanded to the radially expanded state once the prosthetic valve reaches the implantation site. It is understood that the prosthetic valves disclosed herein may be used with a variety of implant delivery apparatuses and can be implanted via various delivery procedures, examples of which will be discussed in more detail later.

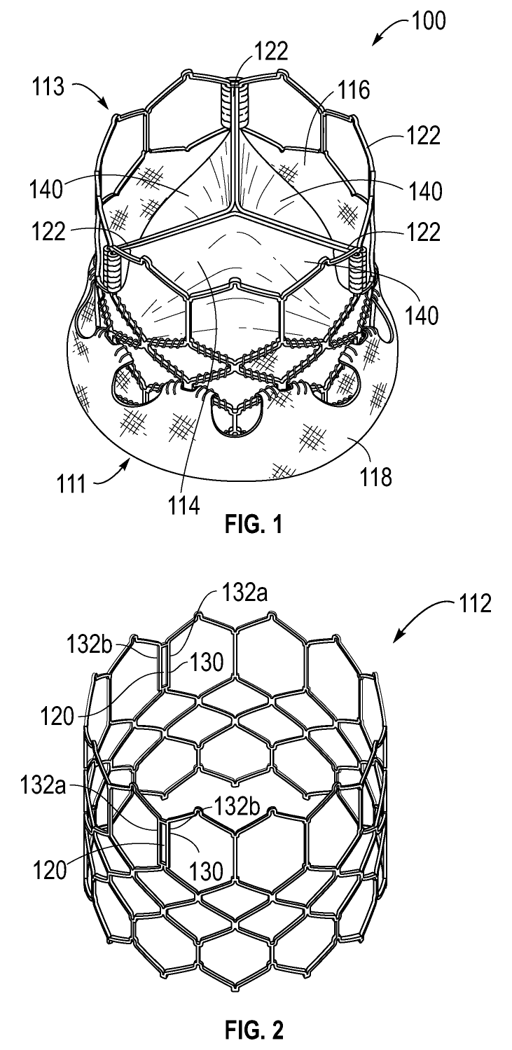

FIG. 1 is a perspective view of an exemplary prosthetic valve 100, according to one example. Any of the prosthetic valves disclosed herein are adapted to be implanted in the native aortic annulus, although in some examples they can be adapted to be implanted in the other native annuluses of the heart (the pulmonary, mitral, and tricuspid valves). The disclosed prosthetic valves also can be implanted within vessels communicating with the heart, including a pulmonary artery (for replacing the function of a diseased pulmonary valve), or the superior vena cava or the inferior vena cava (for replacing the function of a diseased tricuspid valve) or various other veins, arteries and vessels of a patient. The disclosed prosthetic valves also can be implanted within a previously implanted prosthetic valve (which can be a prosthetic surgical valve or a prosthetic transcatheter heart valve) in a valve-in-valve procedure.

In some examples, the disclosed prosthetic valves can be implanted within a docking or anchoring device that is implanted within a native heart valve or a vessel. For example, in one example, the disclosed prosthetic valves can be implanted within a docking device implanted within the pulmonary artery for replacing the function of a diseased pulmonary valve, such as disclosed in U.S. Patent Publication No. 2017/0231756, which is incorporated by reference herein. In some examples, the disclosed prosthetic valves can be implanted within a docking device implanted within or at the native mitral valve, such as disclosed in PCT Publication No. WO2020/247907, which is incorporated herein by reference. In some examples, the disclosed prosthetic valves can be implanted within a docking device implanted within the superior or inferior vena cava for replacing the function of a diseased tricuspid valve, such as disclosed in U.S. Patent Publication No. 2019/0000615, which is incorporated herein by reference.

As shown in FIG. 1, the prosthetic heart valve 100 includes a stent or frame 112, a valvular structure 114, an inner skirt 116, and an outer skirt 118. The prosthetic heart valve 100 includes an inflow end 111 and an outflow end 113. The terms “inflow” and “outflow” are related to the normal direction of blood flow (for example, antegrade blood flow) through the prosthetic valve 100. For example, the valvular structure 114 can allow blood flow through the prosthetic heart valve 100 in a direction from the inflow end 111 to the outflow end 113 and prevent the reverse flow (for example, prevent flow in a direction from the outflow end 113 to the inflow end 111).

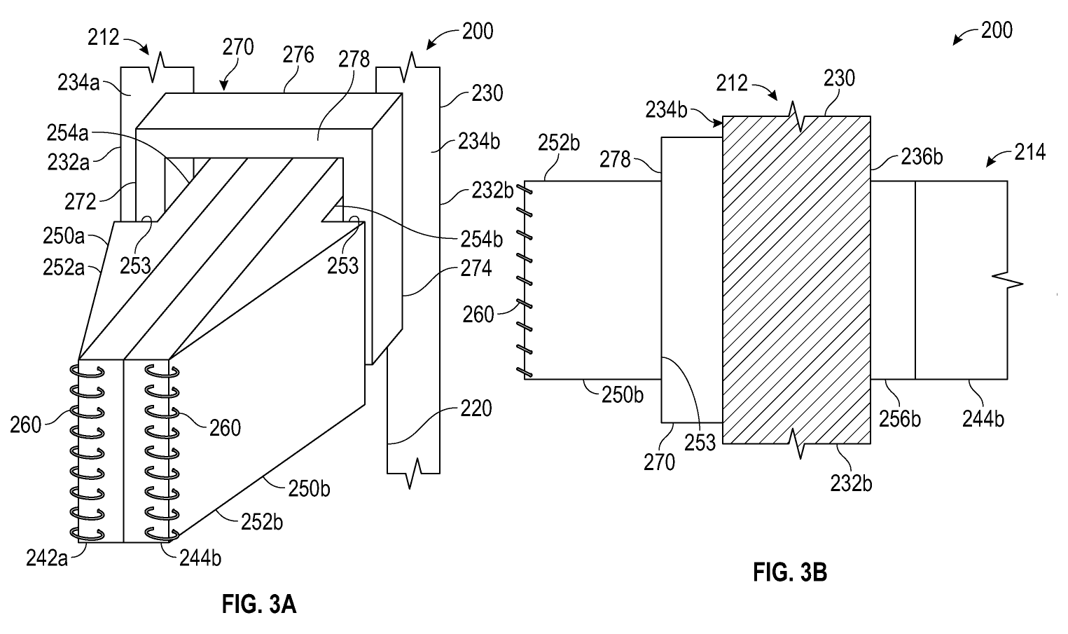

Now referring to FIG. 2, a perspective view of the frame 112 of the prosthetic heart valve 100 is shown. The frame 112 is an annular structure comprising a plurality of circumferentially spaced commissure windows 120 (which are also referred to herein as “frame openings”). The illustrated frame 112 comprises three circumferentially spaced commissure windows 120. However, some examples of prosthetic heart valves can comprise any number of commissure windows 120. Each of the plurality of commissure windows 120 comprises an axially extending slot in the frame 112. The plurality of commissure windows 120 facilitate the mounting of the valvular structure 114 to the frame 112.

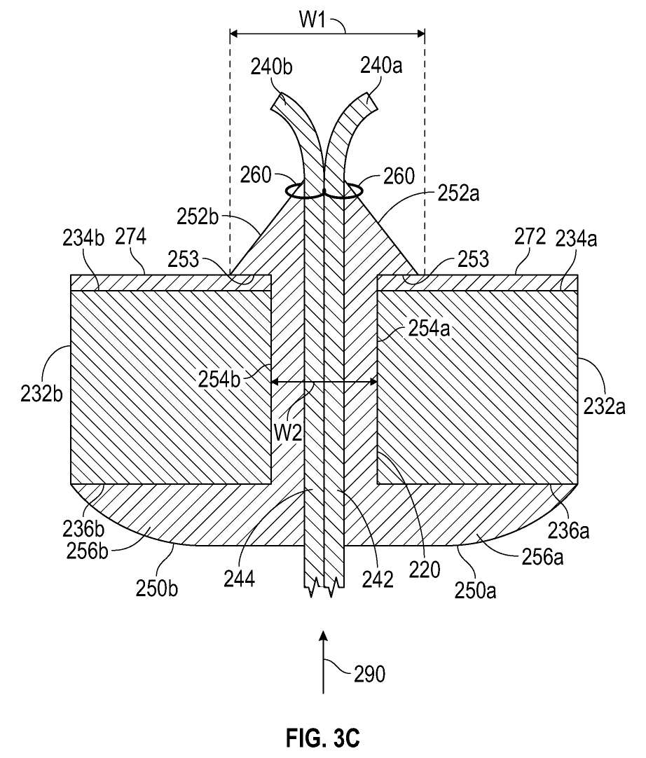

Each commissure window 120 is defined by a corresponding commissure window frame portion 130. Each commissure window frame portion 130 comprises a pair of first and second commissure support portions 132a and 132b (which are also referred to herein as “commissure support struts” or “support posts”). Each of the first and second commissure support portions 132a and 132b comprises an axially extending strut having an inner surface (FIG. 3C) facing radially inwards of the prosthetic heart valve 100 and an outer surface (FIG. 3C) facing radially outwards of the prosthetic heart valve 100. Each pair of commissure support portions 132a and 132b defines a corresponding commissure window 120 circumferentially therebetween.

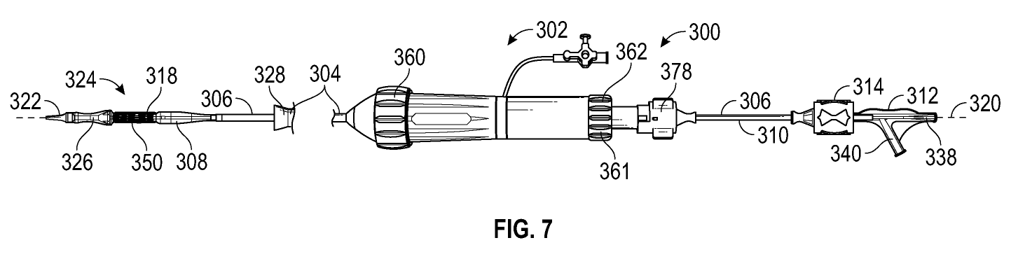

The frame 112 can be made of any of various suitable plastically-expandable materials (for example, stainless steel, etc.) or self-expanding materials (for example, Nitinol) as known in the art. When constructed of a plastically-expandable material, the frame 112 (and thus the prosthetic heart valve 100) can be crimped to a radially compressed state on a delivery catheter (FIG. 7) and then expanded inside a patient by an inflatable balloon or equivalent expansion mechanism. When constructed of a self-expandable material, the frame 112 (and thus the prosthetic heart valve 100) can be crimped to a radially compressed state and restrained in the compressed state by insertion into a sheath or equivalent mechanism of a delivery catheter. Once inside the body, the prosthetic heart valve 100 can be advanced from the delivery sheath, which allows the prosthetic heart valve 100 to expand to its functional size.

Suitable plastically-expandable materials that can be used to form the frames disclosed herein (for example, the frame 112) include, metal alloys, polymers, or combinations thereof. Example metal alloys can comprise one or more of the following: nickel, cobalt, chromium, molybdenum, titanium, or other biocompatible metal. In some examples, the frame 112 can comprise stainless steel. In some examples, the frame 112 can comprise cobalt-chromium. In some examples, the frame 112 can comprise nickel-cobalt-chromium. In some examples, the frame 112 can comprise a nickel-cobalt-chromium-molybdenum alloy, such as MP35N™ (tradename of SPS Technologies), which is equivalent to UNS R30035 (covered by ASTM F562-02). MP35N™/UNS R30035 comprises 35% nickel, 35% cobalt, 20% chromium, and 10% molybdenum, by weight.

Now referring back to FIG. 1, the illustrated valvular structure 114 includes three flexible leaflets 140, collectively forming a leaflet structure or leaflet assembly arranged to collapse in a tricuspid arrangement. However, some examples of the valvular structure 114 can include any plurality of leaflets 140 arranged in any leaflet structure or arrangement. The leaflets 140 of the valvular structure 114 are secured to one another at their adjacent sides to form commissures 122. The valvular structure 114 is secured to the frame 112, for example, by inserting a portion of each commissure 122 through a respective one of the plurality of commissure windows 120 and suturing the commissure 122 to the respective one of the commissure window frame portions 130.

Each of the leaflets 140 can be formed of pericardial tissue (for example, bovine pericardial tissue), biocompatible synthetic materials, or various other suitable natural or synthetic materials as known in the art and described in U.S. Pat. No. 6,730,118, which is incorporated by reference herein. In some examples, each of the leaflets 40 can be formed of a compressible material.

The inner skirt 116 is disposed within and secured to the frame 112 (for example, radially between the valvular structure 114 and the frame 112). The inner skirt 116 is configured to assist in forming a good seal between the prosthetic heart valve 100 and the native annulus by blocking the flow of blood through open cells of the frame 112. The outer skirt 118 is disposed around and secured to the frame 112. When the prosthetic heart valve 100 is deployed, the outer skirt 118 can fill in gaps between the frame 112 and the surrounding native annulus to assist in forming a good, fluid-tight seal between the prosthetic heart valve 100 and the native annulus. The outer skirt 118 therefore cooperates with the inner skirt 116 to avoid perivalvular leakage after implantation of the prosthetic valve 100.

Any one of the inner skirt 116 and the outer skirt 118 can be wholly or partly formed of any suitable biological material, synthetic material (for example, any of various polymers), or combinations thereof. In some examples, the inner skirt 116 and/or the outer skirt 118 can comprise a fabric having interlaced yarns or fibers, such as in the form of a woven, braided, or knitted fabric. In some examples, the fabric can have a plush nap or pile. Exemplary fabrics having a plus nap or pile include velour, velvet, velveteen, corduroy, terrycloth, fleece, etc. In some examples, the inner skirt 116 and/or the outer skirt 118 can comprise a fabric without interlaced yarns or fibers or randomly interlaced yarns or fibers, such as felt or an electrospun fabric. Exemplary materials that can be used for forming such fabrics (with or without interlaced yarns or fibers) include, without limitation, polyethylene (PET), ultra-high molecular weight polyethylene (UHMWPE), polytetrafluoroethylene (PTFE), expanded polytetrafluoroethylene (ePTFE), polyamide etc. In some examples, the inner skirt 116 and/or the outer skirt 118 can comprise a non-textile or non-fabric material, such as a film made from any of a variety of polymeric materials, such as PTFE, PET, polypropylene, polyamide, polyetheretherketone (PEEK), polyurethane (such as thermoplastic polyurethane (TPU)), etc. In some examples, the inner skirt 116 and/or the outer skirt 118 can comprise a sponge material or foam, such as polyurethane foam. In some examples, the inner skirt 116 and/or the outer skirt 118 can comprise natural tissue, such as pericardium (for example, bovine pericardium, porcine pericardium, equine pericardium, or pericardium from other sources).

Further details of the prosthetic heart valve 100 and its variants are described in U.S. Patent No. 9,393,110, which is incorporated by reference herein in its entirety.

FIG. 3A is a perspective view of a portion of an exemplary prosthetic heart valve 200. For convenience, similar reference numbers shown in FIGS. 1-2 can describe components illustrated in FIG. 3A. For example, frame 212 can be similar to frame 112, commissure window 220 can be similar to commissure window 120, commissure window frame portion 230 can be similar to commissure window frame portion 130, and commissure support portions 232a and 232b can be similar to commissure support portions 132a and 132b. However, it should be understood that this numbering convention is merely for convenience and is not intended to limit and/or exclude any claim scope.

As shown, the prosthetic heart valve 200 comprises a valvular structure 214 mounted to the frame 212. The portion of the frame 212 illustrated in FIG. 3A comprises a commissure window 220 and a commissure window frame portion 230 defining the commissure window 220. The commissure window frame portion 230 comprises first and second commissure support portions 232a and 232b extending in an axial direction of the frame. Each of the first and second commissure support portions 232a and 232b comprises a radially facing outer surface 234a and 234b, respectively. Each of the first and second commissure support portions 232a and 232b further comprises a radially facing inner surface 236a and 236b (best seen in FIG. 3C), respectively.

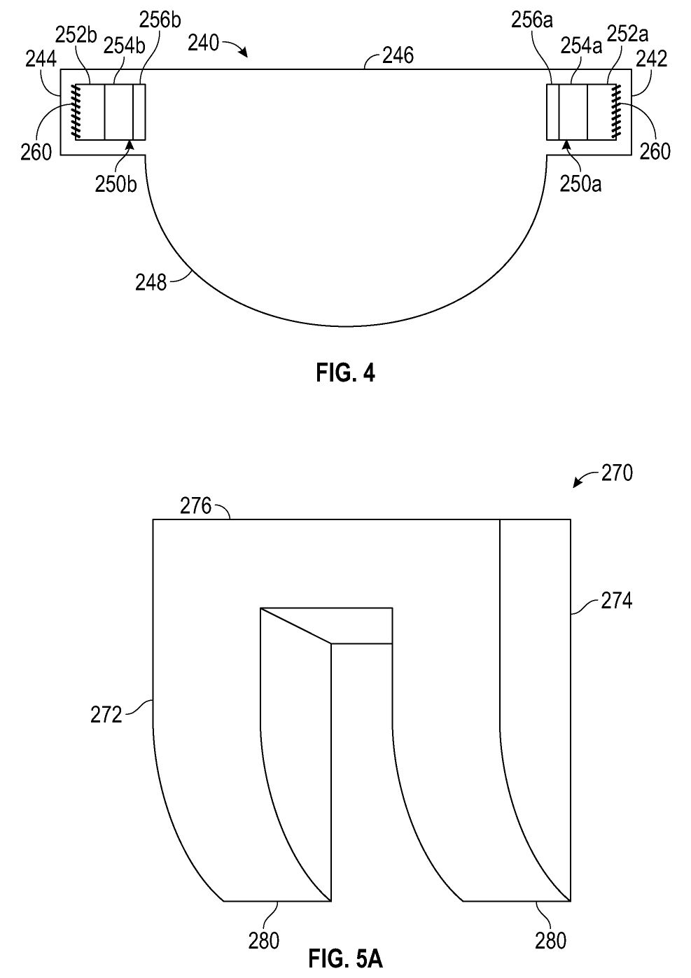

The portion of the valvular structure 214 illustrated in FIGS. 3A-3C comprises first and second leaflets 240a and 240b. Even though only portions of two leaflets are shown, it should be understood that the valvular structure 214 can comprise any number of leaflets. For example, the valvular structure 214 can, similar to the valvular structure 114 shown in FIG. 1, comprise three leaflets arranged in a tricuspid arrangement. The illustrated portion of the first leaflet 240a comprises a first commissure tab 242 (which is also referred to herein as a “tab” and/or a “side tab”). The illustrated portion of the second leaflet 240b comprises a second commissure tab 244 (which is also referred to herein as a “tab” and/or a “side tab”). As shown later, for example, in FIG. 4, each of the first leaflet 240a and second leaflet 240b can each have a first and second commissure tabs 242 and 244 disposed on opposite sides of the respective leaflets 240a and 240b. Each commissure tab 242, 244 of each leaflet 240 (for example, the first leaflet 240a) of the prosthetic heart valve 200 can be paired to with an adjacent commissure tab 242, 244 of an adjacent leaflet 240 (for example, the second leaflet 240b) to form a commissure secured to a respective commissure window frame portion 230. In FIGS. 3A-3C, the first commissure tab 242 of the first leaflet 240a is paired with the second commissure tab 244 of the second leaflet 240b to form a commissure extending at least partially through the commissure window 220 of the frame 212.

One exemplary difference between the prosthetic heart valve 200 and the prosthetic heart valve 100 illustrated in FIG. 1 is that the commissure tabs of the valvular structure 214 need not be sutured to the frame 212. Instead, the valvular structure 214 is secured to the frame 212 using first and second clipping members 250a and 250b (which are also referred to herein as “first and second clips”). In some examples, the first and second clipping members 250a, 250b can be in the form of plugs, pins, or strips that can be attached to the leaflets 240. The first clipping member 250a is secured to the first commissure tab 242a of the first leaflet 240a and the second clipping member 250b is secured to the second commissure tab 244b of the second leaflet 240b. When the first and second clipping members 250a and 250b are pressed together—sandwiching the corresponding commissure tabs 242a and 244b of the first and second leaflets 240a and 240b therebetween—and inserted at least partially through the commissure window 220, the first and second clipping members 250a and 250b form snap-fit connections with the first and second commissure support portions 232a and 232b, respectively, to secure the commissure to the frame 212.

Each of the first clipping member 250a and the second clipping member 250b comprises a wedge member having an outer portion 252a and 252b, an intermediate portion 254a and 254b, and an inner portion 256a and 256b, respectively. For example, the first clipping member 250a can comprise a first outer portion 252a (which is also referred to herein as a “first wedge”), a first intermediate portion 254a, and a first inner portion 256a (which is also referred to herein as a “first flange”), and the second clipping member 250b can comprise a second outer portion 252b (which is also referred to herein as a “second wedge”), a second intermediate portion 254b, and a second inner portion 256b (which is also referred to herein as a “second flange”). When the first clipping member 250a is clipped to the frame 212, the outer portion 252a, the intermediate portion 254a, and the inner portion 256a are aligned in the radial direction of the prosthetic heart valve 200. Similarly, when the second clipping member 250b is clipped to the frame 212, the outer portion 252b, the intermediate portion 254b, and the inner portion 256b are aligned in the radial direction of the prosthetic heart valve 200. Each of the outer portions 252a and 252b comprises a wedge tapering in a radially extending outward direction of the prosthetic heart valve 200 (in other words, in a direction away from the respective intermediate portions 254a and 254b and/or inner portions 256a and 256b). The sloped portion of each wedge can facilitate the insertion of its corresponding clipping member through the commissure window 220 to form the corresponding snap-fit connection, while the flat base portion 253 of each wedge can prevent its corresponding clipping member from being dislodged from the commissure window 220. Each intermediate portion 254a and 254b extends through the commissure window 220 when the first and second clipping members 250a and 250b engage their respective commissure support portions 232a and 232b.

As shown, the first clipping member 250a is secured to the first commissure tab 242a of the first leaflet 240a and the second clipping member 250b is secured to the second commissure tab 244b of the second leaflet 240b using, for example, sutures 260. As further shown, in some examples, the sutures 260 connect the outer portions 252a and 252b of the clipping members 250a and 250b to their respective commissure tabs 242a and 244b. By positioning the sutures 260 on the outside of the frame 212 and away from any cycling forces acting on the leaflets 240a and 240b, the load on the sutures 260 can be reduced, thereby improving the durability of the prosthetic heart valve 200. However, it should be understood that the clipping members 250a and 250b can be secured to the commissure tabs 242a and 242b in any manner (for example, using mechanical fasteners, adhesives, ultrasonic welds, etc.) and, furthermore, that any portion of the clipping members 250a and 250b can be coupled to any portion of the commissure tabs 242a and 244b with sutures or other connection means.

In some examples, each leaflet 240 can be integrally formed with a clipping member 250 on each commissure tab 242, 244. For example, the leaflets 240a and 240b and the clipping members 250a and 250b can be made from a synthetic material (such as any of various polymers), and each leaflet and corresponding clipping members can molded to form a unitary body that includes the leaflet and clipping members.

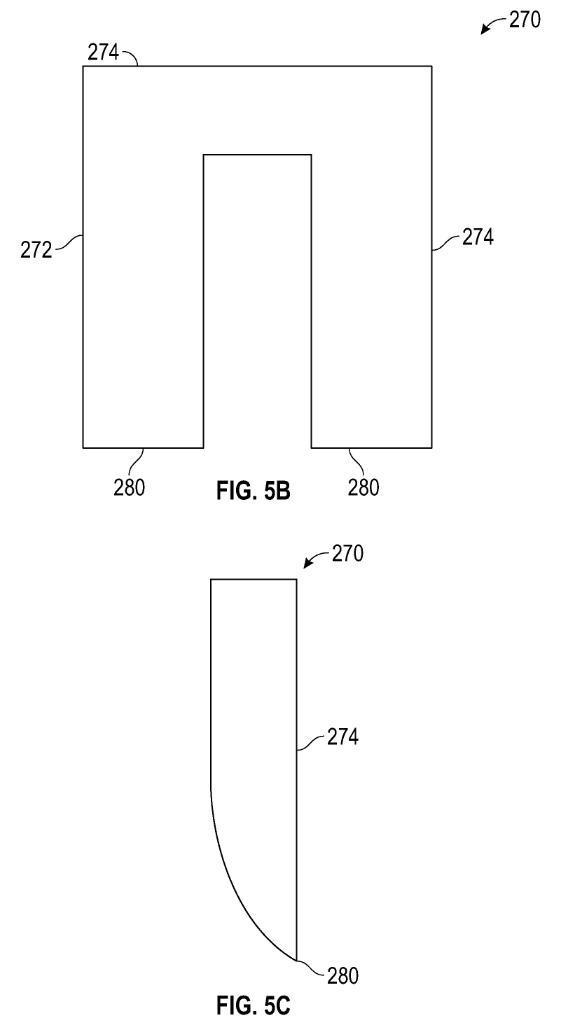

As shown, the prosthetic heart valve 200 can optionally include a shim 270 at each commissure. As best shown in FIG. 5A, the shim 270 can comprise a flat, U-shaped plate comprising a first leg 272, a second leg 274, and a cross-piece 276 connecting the two. When disposed radially between the first commissure support portion 232a and the first clipping member 250a and/or radially between the second commissure support portion 232b and the second clipping member 250b, the shim 270 can fill any gaps in case tolerances between the clipping members and commissure support portions do not allow for a tight fit. The shim 270 further comprises a radially facing outer surface 278 that faces in an outward direction of the prosthetic heart valve 200 when the shim 270 is inserted between the clipping members and commissure support portions. However, since the shim 270 is optional, it should be understood that some examples of the prosthetic heart valve 200 can lack this component.

In some examples, the first clipping member 250a and the second clipping member 250b can be made from a variety of polymeric materials, such as PTFE, PET, polypropylene, polyamide, polyetheretherketone (PEEK), polyurethane (such as thermoplastic polyurethane (TPU)), silicone etc. In some examples, the first clipping member 250a and the second clipping member 250b can be made from a metal, such as nitinol, steel, aluminum, titanium, etc. In some examples, the first clipping member 250a and the second clipping member 250b can be made from a different material than the first and second leaflets 240a and 240b of the valvular structure 214. In some examples, the first and second clipping members 250a and 250b are distinct components that are separately formed from the first and second leaflets 240a and 240b.

In some examples, since it can take less time and/or be easier to attach the valvular structure 214 to the frame 212 using clipping members 250a and 250b than sutures, the clipping members 250a and 250b can beneficially simplify the process of assembling the prosthetic heart valve 200. Additionally or alternatively, in some examples, the clipping members 250a and 250b can beneficially result in a durable connection between the frame 212 and the valvular structure 214 that can withstand forces acting on the prosthetic heart valve 200 during cycling. In some examples, no sutures are used to secure each of the commissures to the frame of the prosthetic heart valve; instead, the holding force of the clipping members 250a, 250b exerted against the commissure support portions 232a, 232b are sufficient to retain the commissures in place within the commissure windows. In some examples, some sutures may be used to secure a pair of commissure tabs to the commissure support portions 232a, 232b.

FIG. 3B is a side view of a portion of the exemplary prosthetic heart valve 200. The left side of the figure corresponds to an outer portion of a commissure of the prosthetic heart valve 200, while the right side of the figure corresponds to an inner portion of the commissure of the prosthetic heart valve 200.

As shown, the second clipping member 250b is inserted through the commissure window 220 such that the outer portion 252b of the second clipping member 250b contacts or engages the outer surface 278 of the shim 270 (more particularly, the outer surface 278 of the second leg 274 of the shim 270). However, in some examples where the shim 270 is not needed, the outer portion 252b of the second clipping member 250b can instead contact or engage the outer surface 234b of the second commissure support portion 232b.

Furthermore, as shown, when the outer portion 252b of the second clipping member 250b is inserted through the commissure window 220, the inner portion 256b of the second clipping member 250b is retained inside the frame 212 such that the inner portion 256b contacts or engages the inner surface 236b of the second commissure support portion 232b. The outer portion 252b and the inner portion 256b sandwich the commissure support portion 232b therebetween to beneficially prevent movement of the second clipping member 250b relative to the commissure support portion 232b.

FIG. 3C is a cross-sectional view of a portion of the exemplary prosthetic heart valve 200. As shown, the inner portions 256aand 256b of the first and second clipping members 250a and 250b can each comprise a flange extending in a circumferential direction of the prosthetic heart valve 200. For example, the first clipping member 250a can comprise a first flange and the second clipping member 250b can comprise a second flange. As further shown, the end portions of the inner portions 256a and 256b can comprise at least partially curved surfaces, but the inner portions 256a and 256b can alternatively comprise a combination of planar surfaces.

The outer portions 252a and 252b and the inner portions 256a and 256b extend at least partially over the outer surfaces 234a and 234b and the inner surfaces 236a and 236b of the respective commissure support portions 232a and 232b. As shown, the inner portions 256a and 256b extend over the inner surfaces 236a and 236b of the first and second commissure support portions 232a and 232b, respectively. As further shown, the outer portions 252a and 252b extend partially over the outer surfaces 234a and 234b of the commissure support portions 232a and 232b. Thus, in the illustrated example, each of the inner portions 256a and 256b is wider in a circumferential direction of the prosthetic heart valve 200 than its corresponding outer portion 252a and 252b. However, it should be understood that the inner portions 256a and 256b and the outer portions 252a and 256b can have any relative ratio of widths or other dimensions relative to each other or relative to the first and second commissure support portions 232a and 232b.

FIG. 4 is a flattened view of a leaflet 240, according to one example. In some examples, where the first leaflet 240a and the second leaflet 240b of the valvular structure 214 are substantially identical, the leaflet 240 can be representative of any leaflet in the valvular structure 214. However, it should be understood that some examples of the valvular structure 214 can comprise non-identical leaflets.

The leaflet 240 in the illustrated configuration comprises first and second commissure tabs 242 and 244 on opposite sides of the leaflet 240, an upper (outflow) free edge 246 extending between the tabs 242 and 244, and a lower (inflow) edge portion 248 extending between the tabs 242 and 244. Although the upper free edge 246 is illustrated as being substantially straight and the lower free edge 248 is illustrated as being curved, some examples of the upper free edge 246 and the lower free edge 248 can have any combination of shapes or curvatures.

As shown, the first clipping member 250a is secured to the first commissure tab 242 using one or more sutures 260. While the suture 260 is shown as coupled only to the outer portion 252a of the first clipping member 250a, any combination of the outer portion 252a, the intermediate portion 254a, and/or the inner portion 256a of the first clipping member 250a can be connected to the commissure tab 242 with sutures and/or other connection means.

Similarly, the outer portion 252b of the second clipping member 250b can be secured to the second commissure tab 244 using one or more sutures 260. In some examples, any combination of the outer portion 252b, the intermediate portion 254b, and/or the inner portion 256b of the second clipping member 250b can be connected to the commissure tab 242 with sutures and/or other connection means.

FIGS. 5A, 5B, and 5C are perspective views, front views, and side views of the exemplary shim 270 of the prosthetic heart valve 200, respectively. As shown, the first leg 272 and the second leg 274 of the shim 270 can taper in a direction away from the cross-piece 276 to define relatively narrow lower ends 280. In some examples, tapering the first leg 272 and/or the second leg 274 can facilitate the insertion of the shim 270 between the frame 212 and the clipping members 250a and 250b, for example, when the shim 270 is inserted along a longitudinal direction of the prosthetic heart valve 200 between the frame 212 and the clipping members 250a and 250b.

In some examples, the shim 270 can be inserted radially between at least one of the clipping members 250a and 250b and the frame 212 after the clipping members 250a and 250b have been snap-fit to the frame 212. In some examples, the shim 270 can be held against the frame 212 prior to the insertion of the clipping members 250a and 250b through the commissure window 220.

A method for assembling the leaflets 240 to the frame 212 of the prosthetic valve can include the following steps. As noted above, the valvular structure 214 can include a plurality of leaflets 240 (for example, three leaflets). Each leaflet 240 can have clipping members 250a, 250b on the commissure tabs 242, 244, respectively. Each commissure tab 242, 244 can be paired with another commissure tab 242, 244 of another leaflet 240. For example, as shown in FIGS. 3A-3C, a first commissure tab 242 of a first leaflet 240a is paired with a second commissure tab 244 of a second leaflet 240b. The commissure tabs 242, 244 are placed side by side and positioned inside the frame 212 near a commissure window 220.

The two commissure tabs 242, 244 (with their respective clipping members 250a, 250b) are then conveyed (for example, pushed) through the commissure window 220 in the radial outward direction, as indicated by arrow 290 in FIG. 3C. As the commissure tabs 242, 244 and the clipping members 250a, 250b are pushed through the commissure window 220, the commissure tabs 242, 244 can be compressed between the clipping members 250a, 250b by virtue of tapered outer portions 252a, 252b sliding against the inner side surfaces of the commissure support portions 232a, 232b. Compression of the commissure tabs 242, 244 reduces the maximum width W1 between the outer portions 252a, 252b so that it is less than the width W2 of the commissure window 220, allowing the commissure tabs 242, 244 and the clipping members 250a, 250b to slide through the commissure window 220. In lieu of or in addition to the commissure tabs 242, 244 being compressible, the outer portions 252a, 252b can be deformable or compressible to reduce the width the W1 to less than the width W2 facilitate insertion of the clipping members 250a, 250b through the commissure window 220. In some examples, the clipping members 250a, 250b can be made from a deformable and resilient polymer, such as polyurethane or silicone, to enable compression of the outer portions 252a, 252b as the clipping members 250a, 250b are conveyed through the commissure window 220.

As the outer portions 252a, 252b of the clipping members 250a, 250b exit the commissure window 220 on the outside of the frame 212, and the intermediate portions 254a, 254b become aligned with the commissure support portions 232a, 232b within the commissure window 220, the commissure tabs 242, 244 and/or the outer portions 252a, 252b can expand in width back to its initial width W1 (which is greater than width W2), allowing the outer portions 252a, 252b to expand away from each other. Once expanded, the outer portion 252a extends over the adjacent outer surface 234a of the commissure support portion 232a and the outer portion 252b extends over the adjacent outer surface 234b of the commissure support portion 232b, preventing the commissure tabs 242, 244 from being pulled back through the window 220 in a direction into the frame 212. The inner portions 256a, 256b extend over adjacent inner surfaces 236a, 236b of the commissure support portions 232a, 232b, respectively, to prevent the commissure tabs 242, 244 and the clipping members 250a, 250b from further travel through the commissure window 220 in the radial outward direction.

The clipping members 250a, 250b can be said to form a snap fit connection with the commissure support portions 232a, 232b by virtue of the commissure tabs 242, 244 and/or the clipping members 250a, 250b deforming to fit through the commissure window 220 and then resiliently expanding in width once the outer portions 252a, 252b are pushed beyond the commissure support portions 232a, 232b to form a connection with the commissure support portions 232a, 232b. The clipping members 250a, 250b can, but need not, produce an audible clicking sound when pushed through the commissure window 220 to form the connection.

After clipping members 250a, 250b are positioned within the commissure window 220, a shim 270 can be inserted into the space between the commissure support portions 232a, 232b and the outer portions 252a, 252b to reduce any gaps or play between adjacent surfaces of the commissure support portions 232a, 232b and the clipping members 250a, 250b in the radial direction. The shim 270 can be inserted into place by sliding the shim 270 in a longitudinal direction (parallel to the central longitudinal axis of the frame 212) along the outside of the frame 212 between the commissure support portions 232a, 232b and the outer portions 252a, 252b of the clipping members 250a, 250b.

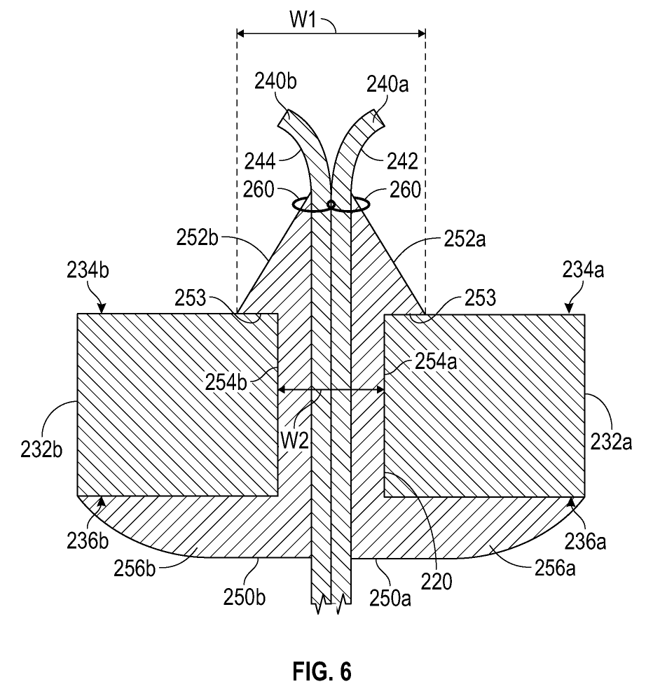

FIG. 6 is a cross-sectional view of a portion of the prosthetic heart valve 200. One exemplary difference between the cross-section shown in FIG. 6 and the cross-sectional view shown in FIG. 3C is that the optional shim 270 is omitted from FIG. 6. Thus, as shown, the outer portion 252a of the first clipping member 250a contacts the outer surface 234a of the first commissure support portion 232a and the outer portion 252b of the second clipping member 250b contacts the outer surface 234b of the first commissure support portion 232b when the clipping members 250a and 250b are snap-fit to the frame 212.

FIG. 7 shows a delivery apparatus 300, according to one example, in the form of a balloon catheter that can be used to implant a prosthetic medical device. In some examples, the delivery apparatus 300 can be used to implant an expandable prosthetic heart valve (for example, the prosthetic heart valve 200 of FIGS. 3-6 and/or any of the other prosthetic heart valves described herein). In some examples, the delivery apparatus 300 is specifically adapted for use in introducing a prosthetic heart valve into a heart.

The delivery apparatus 300 in the illustrated example of FIG. 7 comprises a handle 302 and a steerable, outer shaft 304 extending distally from the handle 302. The delivery apparatus 300 can further comprise an intermediate shaft 306 (which also may be referred to as a balloon shaft) that extends proximally from the handle 302 and distally from the handle 302, the portion extending distally from the handle 302 also extending coaxially through the outer shaft 304. Additionally, the delivery apparatus 300 can further comprise an inner shaft 308 extending distally from the handle 302 coaxially through the intermediate shaft 306 and the outer shaft 304 and proximally from the handle 302 coaxially through the intermediate shaft 306.

The outer shaft 304 and the intermediate shaft 306 can be configured to translate (for example, move) longitudinally, along a central longitudinal axis 320 of the delivery apparatus 300, relative to one another to facilitate delivery and positioning of a prosthetic heart valve at an implantation site in a patient’s body.

The intermediate shaft 306 can include a proximal end portion 310 that extends proximally from a proximal end of the handle 302, to an adaptor 312. A rotatable knob 314 can be mounted on the proximal end portion 310 and can be configured to rotate the intermediate shaft 306 around the central longitudinal axis 320 and relative to the outer shaft 304.

The adaptor 312 can include a first port 338 configured to receive a guidewire therethrough and a second port 340 configured to receive fluid (for example, inflation fluid) from a fluid source. The second port 340 can be fluidly coupled to an inner lumen of the intermediate shaft 306.

The intermediate shaft 306 can further include a distal end portion that extends distally beyond a distal end of the outer shaft 304 when a distal end of the outer shaft 304 is positioned away from an inflatable catheter balloon 318 (which also referred to herein as a “balloon”) of the delivery apparatus 300. A distal end portion of the inner shaft 308 can extend distally beyond the distal end portion of the intermediate shaft 306.

The catheter balloon 318 can be coupled to the distal end portion of the intermediate shaft 306.

In some examples, a distal end of the catheter balloon 318 can be coupled to a distal end of the delivery apparatus 300, such as to a nose cone 322 (as shown in FIG. 7), or to an alternate component at the distal end of the delivery apparatus 300 (for example, a distal shoulder). An intermediate portion of the catheter balloon 318 can overlay a valve mounting portion 324 of a distal end portion of the delivery apparatus 300 and a distal end portion of the catheter balloon 318 can overly a distal shoulder 326 of the delivery apparatus 300. The valve mounting portion 324 and the intermediate portion of the catheter balloon 318 can be configured to receive a prosthetic heart valve in a radially compressed state. For example, as shown schematically in FIG. 7, a prosthetic heart valve 350 (which can be one of the prosthetic heart valves described herein) can be mounted around the catheter balloon 318, at the valve mounting portion 324 of the delivery apparatus 300.

The balloon shoulder assembly, including the distal shoulder 326, is configured to maintain the prosthetic heart valve 350 (or other prosthetic medical device) at a fixed position on the catheter balloon 318 during delivery through the patient’s vasculature.

The outer shaft 304 can include a distal tip portion 328 mounted on its distal end. The outer shaft 304 and the intermediate shaft 306 can be translated axially relative to one another to position the distal tip portion 328 adjacent to a proximal end of the valve mounting portion 324, when the prosthetic heart valve 350 is mounted in the radially compressed state on the valve mounting portion 324 (as shown in FIG. 7) and during delivery of the prosthetic heart valve to the target implantation site. As such, the distal tip portion 328 can be configured to resist movement of the prosthetic heart valve 350 relative to the catheter balloon 318 proximally, in the axial direction, relative to the catheter balloon 318, when the distal tip portion 328 is arranged adjacent to a proximal side of the valve mounting portion 324.

An annular space can be defined between an outer surface of the inner shaft 308 and an inner surface of the intermediate shaft 306 and can be configured to receive fluid from a fluid source via the second port 340 of the adaptor 312. The annular space can be fluidly coupled to a fluid passageway formed between the outer surface of the distal end portion of the inner shaft 308 and an inner surface of the catheter balloon 318. As such, fluid from the fluid source can flow to the fluid passageway from the annular space to inflate the catheter balloon 318 and radially expand and deploy the prosthetic heart valve 350.

An inner lumen of the inner shaft can be configured to receive a guidewire therethrough, for navigating the distal end portion of the delivery apparatus 300 to the target implantation site.

The handle 302 can include a steering mechanism configured to adjust the curvature of the distal end portion of the delivery apparatus 300. In the illustrated example, for example, the handle 302 includes an adjustment member, such as the illustrated rotatable knob 360, which in turn is operatively coupled to the proximal end portion of a pull wire. The pull wire can extend distally from the handle 302 through the outer shaft 304 and has a distal end portion affixed to the outer shaft 304 at or near the distal end of the outer shaft 304. Rotating the knob 360 can increase or decrease the tension in the pull wire, thereby adjusting the curvature of the distal end portion of the delivery apparatus 300. Further details on steering or flex mechanisms for the delivery apparatus can be found in U.S. Patent No. 9,339,384, which is incorporated by reference herein.

The handle 302 can further include an adjustment mechanism 361 including an adjustment member, such as the illustrated rotatable knob 362, and an associated locking mechanism including another adjustment member, configured as a rotatable knob 378. The adjustment mechanism 361 is configured to adjust the axial position of the intermediate shaft 306 relative to the outer shaft 304 (for example, for fine positioning at the implantation site). Further details on the delivery apparatus 300 can be found in PCT Publication No. WO2022/046585, which is incorporated by reference herein.

### Delivery Techniques

For implanting a prosthetic valve within the native aortic valve via a transfemoral delivery approach, the prosthetic valve is mounted in a radially compressed state along the distal end portion of a delivery apparatus. The prosthetic valve and the distal end portion of the delivery apparatus are inserted into a femoral artery and are advanced into and through the descending aorta, around the aortic arch, and through the ascending aorta. The prosthetic valve is positioned within the native aortic valve and radially expanded (for example, by inflating a balloon, actuating one or more actuators of the delivery apparatus, or deploying the prosthetic valve from a sheath to allow the prosthetic valve to self-expand). Alternatively, a prosthetic valve can be implanted within the native aortic valve in a transapical procedure, whereby the prosthetic valve (on the distal end portion of the delivery apparatus) is introduced into the left ventricle through a surgical opening in the chest and the apex of the heart and the prosthetic valve is positioned within the native aortic valve. Alternatively, in a transaortic procedure, a prosthetic valve (on the distal end portion of the delivery apparatus) is introduced into the aorta through a surgical incision in the ascending aorta, such as through a partial J-sternotomy or right parasternal mini-thoracotomy, and then advanced through the ascending aorta toward the native aortic valve.

For implanting a prosthetic valve within the native mitral valve via a transseptal delivery approach, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, into the right atrium, across the atrial septum (through a puncture made in the atrial septum), into the left atrium, and toward the native mitral valve. Alternatively, a prosthetic valve can be implanted within the native mitral valve in a transapical procedure, whereby the prosthetic valve (on the distal end portion of the delivery apparatus) is introduced into the left ventricle through a surgical opening in the chest and the apex of the heart and the prosthetic valve is positioned within the native mitral valve.

For implanting a prosthetic valve within the native tricuspid valve, the prosthetic valve is mounted in a radially compressed state along the distal end portion of a delivery apparatus. The prosthetic valve and the distal end portion of the delivery apparatus are inserted into a femoral vein and are advanced into and through the inferior vena cava, and into the right atrium, and the prosthetic valve is positioned within the native tricuspid valve. A similar approach can be used for implanting the prosthetic valve within the native pulmonary valve or the pulmonary artery, except that the prosthetic valve is advanced through the native tricuspid valve into the right ventricle and toward the pulmonary valve/pulmonary artery.

Another delivery approach is a transatrial approach whereby a prosthetic valve (on the distal end portion of the delivery apparatus) is inserted through an incision in the chest and an incision made through an atrial wall (of the right or left atrium) for accessing any of the native heart valves. Atrial delivery can also be made intravascularly, such as from a pulmonary vein. Still another delivery approach is a transventricular approach whereby a prosthetic valve (on the distal end portion of the delivery apparatus) is inserted through an incision in the chest and an incision made through the wall of the right ventricle (typically at or near the base of the heart) for implanting the prosthetic valve within the native tricuspid valve, the native pulmonary valve, or the pulmonary artery.

In all delivery approaches, the delivery apparatus can be advanced over a guidewire previously inserted into a patient’s vasculature. Moreover, the disclosed delivery approaches are not intended to be limited. Any of the prosthetic valves disclosed herein can be implanted using any of various delivery procedures and delivery devices known in the art.

### Sterilization

Any of the systems, devices, apparatuses, etc. herein can be sterilized (for example, with heat/thermal, pressure, steam, radiation, and/or chemicals, etc.) to ensure they are safe for use with patients, and any of the methods herein can include sterilization of the associated system, device, apparatus, etc. as one of the steps of the method. Examples of heat/thermal sterilization include steam sterilization and autoclaving. Examples of radiation for use in sterilization include, without limitation, gamma radiation, ultra-violet radiation, and electron beam. Examples of chemicals for use in sterilization include, without limitation, ethylene oxide, hydrogen peroxide, peracetic acid, formaldehyde, and glutaraldehyde. Sterilization with hydrogen peroxide may be accomplished using hydrogen peroxide plasma, for example.

### Examples

In view of the above-described implementations of the disclosed subject matter, this application discloses the additional examples enumerated below. It should be noted that one feature of an example in isolation or more than one feature of the example taken in combination and, optionally, in combination with one or more features of one or more further examples are further examples also falling within the disclosure of this application.

Example 1. A prosthetic heart valve can include a frame with first and second commissure support portions defining a frame opening therebetween and a leaflet assembly with a first leaflet including a first commissure tab, a second leaflet including a second commissure tab, a first wedge coupled to the first commissure tab of the first leaflet, and a second wedge coupled to the second commissure tab of the second leaflet, wherein the first and second wedges and the first and second commissure tabs can extend at least partially through the frame opening, the first wedge can be coupled to the first commissure support portion, and the second wedge can be coupled to the second commissure support portion.

Example 2. The prosthetic heart valve of any example herein, particularly Example 1, wherein the first and second wedges can form snap-fit connections with the first and second commissure support portions, respectively.

Example 3. The prosthetic heart valve of any example herein, particularly any one of Examples 1-2, wherein the leaflet assembly can further comprise a first flange and a first intermediate portion disposed between the first wedge and the first flange.

Example 4. The prosthetic heart valve of any example herein, particularly Example 3, wherein the first wedge can contact an outer surface of the first commissure support portion, the first flange can contact an inner surface of the first commissure support portion, and the first intermediate portion can extend through the frame opening.

Example 5. The prosthetic heart valve of any example herein, particularly any one of Examples 3-4, wherein the first wedge can taper away from the first intermediate portion and the first flange.

Example 6. The prosthetic heart valve of any example herein, particularly any one of Examples 1-5, wherein the leaflet assembly can further comprise a second flange and a second intermediate portion disposed between second wedge and the second flange.

Example 7. The prosthetic heart valve of any example herein, particularly Example 6, wherein the second wedge can contact an outer surface of the second commissure support portion, the second flange can contact an inner surface of the second commissure support portion, and the second intermediate portion can extend through the frame opening.

Example 8. The prosthetic heart valve of any example herein, particularly any one of Examples 6-7, wherein the second wedge can taper away from the second intermediate portion and the second flange.

Example 9. A prosthetic heart valve can include an annular frame and a valvular structure including a plurality of leaflets, wherein each leaflet can have opposing commissure tabs, wherein a clip can be secured to each commissure tab, and wherein each clip can be secured to a portion of the frame with a snap-fit connection.

Example 10. The prosthetic heart valve of any example herein, particularly Example 9, wherein each clip can be sutured to a corresponding commissure tab.

Example 11. The prosthetic heart valve of any example herein, particularly any one of Examples 9-10, wherein each clip can comprise an outer portion configured to extend over an outer surface of the portion of the frame.

Example 12. The prosthetic heart valve of any example herein, particularly Example 11, wherein the outer portion of each clip can comprise a wedge tapering away from the outer surface of the portion of the frame.

Example 13. The prosthetic heart valve of any example herein, particularly any one of Examples 11-12, wherein the outer portion of each clip can be secured to a corresponding commissure tab.

Example 14. The prosthetic heart valve of any example herein, particularly Example 13, wherein the outer portion of each clip can be sutured to the corresponding commissure tab.

Example 15. The prosthetic heart valve of any example herein, particularly any one of Examples 9-14, wherein each clip can comprise an inner portion configured to extend over an inner surface of the portion of the frame.

Example 16. A valvular structure for a prosthetic heart valve can include a first leaflet with a first commissure tab disposed on a first side of the first leaflet, a second leaflet with a second commissure tab disposed on a second side of the second leaflet. a first clip coupled to the first commissure tab of the first leaflet, wherein the first clip can be configured to clip onto a first commissure support portion of a frame of the prosthetic heart valve; and a second clip coupled to the second commissure tab of the second leaflet, wherein the second clip can be configured to clip onto a second commissure support portion of the frame of the prosthetic heart valve.

Example 17. The valvular structure of any example herein, particularly Example 16, wherein the first and second clips can be made of a polymer.

Example 18. The valvular structure of any example herein, particularly any one of Examples 16-17, wherein the first and second clips cab be made of nitinol.

Example 19. The valvular structure of any example herein, particularly any one of Examples 16-18, wherein the first and second leaflets can be made of a first material and the first and second clips can be made of a second material.

Example 20. The valvular structure of any example herein, particularly any one of Examples 16-19, wherein the first and second leaflets can be made of pericardium.

Example 21. The valvular structure of any example herein, particularly any one of Examples 16-20, wherein the first and second leaflets can be made of a compressible material.

Example 22. A method of assembling a prosthetic valve can include coupling a first clip to a first commissure tab of a first leaflet, coupling a second clip to a second commissure tab of a second leaflet, and inserting the first commissure tab of the first leaflet and the second commissure tab of the second leaflet through a commissure window of a prosthetic heart valve frame such that the first commissure tab and the second commissure tab are disposed between the first clip and the second clip.

Example 23. The method of claim 22, which can further comprise, after inserting the first commissure tab of the first leaflet and the second commissure tab of the second leaflet through the commissure window, inserting a shim radially between the prosthetic heart valve frame and at least one of the first clip and the second clip.

Example 24. A valvular structure of any example herein, particularly any one of examples 1-23, wherein the valvular structure can be sterilized.

The features described herein with regard to any example can be combined with other features described in any one or more of the other examples, unless otherwise stated. For example, any one or more of the features of one prosthetic heart valve can be combined with any one or more features of another prosthetic heart valve. In some examples, any one or more features of one leaflet assembly can be combined with any one or more features of another leaflet assembly.

In view of the many possible ways in which the principles of the disclosure may be applied, it should be recognized that the illustrated configurations depict examples of the disclosed technology and should not be taken as limiting the scope of the disclosure nor the claims. Rather, the scope of the claimed subject matter is defined by the following claims and their equivalents.

We claim:

1. A prosthetic heart valve comprising:
a frame comprising first and second commissure support portions defining a frame opening therebetween; and
a leaflet assembly comprising:
a first leaflet comprising a first commissure tab;
a second leaflet comprising a second commissure tab;
a first wedge coupled to the first commissure tab of the first leaflet; and
a second wedge coupled to the second commissure tab of the second leaflet,
wherein the first and second wedges and the first and second commissure tabs extend at least partially through the frame opening, the first wedge is coupled to the first commissure support portion, and the second wedge is coupled to the second commissure support portion.

2. The prosthetic heart valve of claim 1, wherein the first and second wedges form snap-fit connections with the first and second commissure support portions, respectively.

3. The prosthetic heart valve of claim 1, wherein the leaflet assembly further comprises a first flange and a first intermediate portion disposed between the first wedge and the first flange.

4. The prosthetic heart valve of claim 3, wherein the first wedge contacts an outer surface of the first commissure support portion, the first flange contacts an inner surface of the first commissure support portion, and the first intermediate portion extends through the frame opening.

5. The prosthetic heart valve of claim 3, wherein the first wedge tapers away from the first intermediate portion and the first flange.

6. The prosthetic heart valve of claim 1, wherein the leaflet assembly further comprises a second flange and a second intermediate portion disposed between second wedge and the second flange.

7. The prosthetic heart valve of claim 6, wherein the second wedge contacts an outer surface of the second commissure support portion, the second flange contacts an inner surface of the second commissure support portion, and the second intermediate portion extends through the frame opening.

8. The prosthetic heart valve of claim 6, wherein the second wedge tapers away from the second intermediate portion and the second flange.

9. A prosthetic heart valve comprising:
an annular frame; and
a valvular structure comprising a plurality of leaflets, each leaflet having opposing commissure tabs, wherein a clip is secured to each commissure tab, and wherein each clip is secured to a portion of the frame with a snap-fit connection.

10. The prosthetic heart valve of claim 9, wherein each clip is sutured to a corresponding commissure tab.

11. The prosthetic heart valve of claim 10, wherein each clip comprises an outer portion configured to extend over an outer surface of the portion of the frame.

12. The prosthetic heart valve of claim 11, wherein the outer portion of each clip comprises a wedge tapering away from the outer surface of the portion of the frame.

13. The prosthetic heart valve of claim 11, wherein the outer portion of each clip is secured to a corresponding commissure tab.

14. The prosthetic heart valve of claim 13, wherein the outer portion of each clip is sutured to the corresponding commissure tab.

15. The prosthetic heart valve of claim 9, wherein each clip comprises an inner portion configured to extend over an inner surface of the portion of the frame.

16. A valvular structure for a prosthetic heart valve, the valvular structure comprising:
a first leaflet comprising a first commissure tab disposed on a first side of the first leaflet;
a second leaflet comprising a second commissure tab disposed on a second side of the second leaflet;
a first clip coupled to the first commissure tab of the first leaflet, wherein the first clip is configured to clip onto a first commissure support portion of a frame of the prosthetic heart valve; and
a second clip coupled to the second commissure tab of the second leaflet, wherein the second clip is configured to clip onto a second commissure support portion of the frame of the prosthetic heart valve.

17. The valvular structure of claim 16, wherein the first and second clips are made of a polymer or nitinol.

18. The valvular structure of claim 16, wherein the first and second leaflets are made of a first material and the first and second clips are made of a second material.

19. The valvular structure of claim 16, wherein the first and second leaflets are made of pericardium or a compressible material.

20. A method of assembling a prosthetic valve, comprising:
coupling a first clip to a first commissure tab of a first leaflet;
coupling a second clip to a second commissure tab of a second leaflet; and
inserting the first commissure tab of the first leaflet and the second commissure tab of the second leaflet through a commissure window of a prosthetic heart valve frame such that the first commissure tab and the second commissure tab are disposed between the first clip and the second clip.

21. The method of claim 20, further comprising, after inserting the first commissure tab of the first leaflet and the second commissure tab of the second leaflet through the commissure window, inserting a shim radially between the prosthetic heart valve frame and at least one of the first clip and the second clip.
